(19) United States
(12) Patent Application Publication
(10) Pub. No.: US 2018/0318332 A1
(43) Pub. Date: Nov. 8, 2018

(54) COMPOSITION AND METHOD FOR THE TREATMENT OF THE SYMPTOMS OF MULTIPLE SCLEROSIS

(71) Applicant: Valery Lipenko, North Richland Hills, TX (US)

(72) Inventor: Valery Lipenko, North Richland Hills, TX (US)

(21) Appl. No.: 15/970,264

(22) Filed: May 3, 2018

Related U.S. Application Data
(60) Provisional application No. 62/500,815, filed on May 3, 2017.

(51) Int. Cl. A61K 31/714; A61K 31/4188; A61K 31/519; A61K 31/593; A61K 31/375; A61K 31/198; A61K 36/74; A61K 36/9066; A61K 31/685; A61K 31/4406; A61P 25/00

(52) U.S. Cl. CPC A61K 31/714; A61K 31/4188; A61K 31/519; A61K 31/593; A61K 31/375; A61P 25/00; A61K 36/74; A61K 36/9066; A61K 31/685; A61K 31/4406; A61K 31/198

(57) ABSTRACT

A compound and method for treating a patient with multiple sclerosis symptoms is disclosed. The compound includes from approximately 1 milligrams to approximately 4 milligrams of vitamin B12, from approximately 150 milligrams to approximately 300 milligrams of biotin, from approximately 1 milligram to approximately 4 milligrams of folate, from approximately 2000 international units to approximately 10,000 international units of vitamin D₃, from approximately 100 milligrams to approximately 1000 milligrams of vitamin C, from approximately 300 milligrams to approximately 1200 milligrams of n-acetylcysteine, from approximately 10 milligrams to approximately 50 milligrams of green coffee bean extract, and from approximately 100 milligrams to approximately 1000 milligrams of turmeric concentrate. The method for treating a patient with multiple sclerosis symptoms includes the steps of providing the compound, apportioning the compound into a set of dosages of the compound, and administering the set of dosages of the compound based on a set of times.

### CROSS-REFERENCE TO RELATED APPLICATION

This application claims priority to U.S. Provisional Application No. 62/500,815, filed May 3, 2017. The above patent application is incorporated herein by reference in its entirety to provide continuity of disclosure.

### FIELD OF THE INVENTION

This invention relates to the treatment of multiple sclerosis. In particular, this invention relates to compositions, systems, apparatus, and methods for treating multiple sclerosis and its symptoms.

### BACKGROUND OF THE INVENTION

Multiple sclerosis (“MS”) is a devastating disease that induces the body's own immune system to attack the central nervous system, including the brain, spinal cord, and optic nerve. Each nerve cell of the central nervous system is connected to a nerve fiber to transmit electrical impulses to a set of axon terminals. The axon terminals then transmit the electrical impulses to other neurons. The nerve fiber is surrounded by a myelin sheath, which is a fatty white substance that insulates the nerve fiber.

Presently, MS is thought to degrade the central nervous system by causing T cells and B cells of the body's immune system to release inflammatory molecules that remove the myelin surrounding the nerve fibers, thereby exposing the nerve fiber. Once exposed, the exposed nerve fibers are degraded in that the electrical impulse signals become distorted or interrupted causing varying symptoms that differ with each patient.

Common symptoms found are: cognitive in nature including depression, anxiety, cognitive impairment, poor sleep, and vertigo; pain including facial pain burning, and aching of extremities and trunk; urinary in nature including urgency, nocturia, incontinence, and hesitancy; sexual in nature including loss of sensation and ability to orgasm, and lower libido; vision problems including loss of vision and double or unstable vision; fatigue including exercise intolerance and exhaustion, which can be aggravated by heat and humidity; sensory in nature including numbness, tingling, and sensitivity to heat and cold; and, mobility-based including poor balance, impaired motor skills and clumsiness.

Two main types of MS have been identified: relapse-remitting MS (“RRMS”) and primary progressive MS (“PPMS”). RRMS generates symptomatic attacks (relapses) which may be unpredictable followed by periods of remission of symptoms that can last months or years. In PPMS, symptoms do not remit and worsen over time.

Despite years of research, the cause remains unknown. Further, treatments for MS are scarce. However, some starting to reveal the complicated interactions between genetics, geography, and a common viral infection that may lie behind the disease. And new treatments, many based on existing drugs, are starting to show promise for the more debilitating progressive form of the disease, which has until recently been largely ignored and forgotten. Leaders in the field are calling on researchers to take more risks and develop new kinds of clinical trial to test different combinations of drugs to treat progressive multiple sclerosis.

An extreme form of treatment that eliminates a patient's immune system and replaces it with fresh stem cells is offering some limited promise. However, many physicians worry that it is too dangerous for widespread use, and this form of treatment is attracting unethical and unlicensed imitators.

Most recently, the U.S. Food and Drug Administration approved a new drug called Ocrevus, known generically as ocrelizumab, manufactured by Genentech, Inc. of South San Francisco, Calif. to treat RRMS. Ocrevus is a monoclonal antibody, which is an engineered immune system molecule that targets B cells of the immune system. However, serious side effects of Ocrevus include increased risk of infections and cancer. Moreover, the cost of this drug is approximately $65,000 per year of treatment, similar to that of the widely used RRMS treatment drug Rebif® (interferon beta-1a) manufactured by Merck Serono of Darmstadt, Germany, also known as EMD Serono, Inc. of Rockland, Mass. Given these expensive and risky treatments, patients are left to manage their disease.

Therefore, there is a need in the art for a composition and method for the treatment of the symptoms of multiple sclerosis that is inexpensive and without harmful side effects. There is a further need in the art for a composition and method for treatment of the symptoms of multiple sclerosis without the need for painful injections.

### SUMMARY

A compound and method for treating symptoms of multiple sclerosis in a patient is disclosed. The compound includes from approximately 1 milligrams to approximately 4 milligrams of vitamin B12, from approximately 150 milligrams to approximately 300 milligrams of biotin, from approximately 1 milligram to approximately 4 milligrams of folate, from approximately 2000 international units to approximately 10,000 international units of vitamin D₃, from approximately 100 milligrams to approximately 1000 milligrams of vitamin C, from approximately 300 milligrams to approximately 1200 milligrams of n-acetylcysteine, from approximately 10 milligrams to approximately 50 milligrams of green coffee bean extract, and from approximately 100 milligrams to approximately 1000 milligrams of turmeric concentrate.

The method for treating a patient with symptoms of multiple sclerosis includes the steps of providing the compound, apportioning the compound into a set of dosages of the compound, and administering the set of dosages of the compound based on a set of times.

### DETAILED DESCRIPTION

A compound for the treatment of symptoms of multiple sclerosis is disclosed. In a preferred embodiment, the compound is a mixture of components that includes vitamin B12, biotin, folate, vitamin D3, vitamin C, n-acetyl cysteine (“NAC”), green coffee bean extract, and turmeric concentrate.

In another embodiment, the compound further includes phosphatidylserine.

In another embodiment, the compound further includes niacin.

In another embodiment, the compound further includes phosphatidylserine and niacin.

In a preferred embodiment, the compound is contained in a capsule. Other suitable delivery means known in the art may be employed.

The components and respective amounts of the components in milligrams (mg) and international units (IU) of the compound are listed in Table 1 below.

TABLE 1

| | Preferred Amount Range | Most Preferred Amount |
|---|---|---|
| Component | (mg) | (mg) |
| Vitamin B12 | Approximately 1 mg to | Approximately 2 mg |
| | Approximately 4 mg | |
| Biotin | Approximately 150 mg to | Approximately 300 mg |
| | Approximately 300 mg | |
| Folate | Approximately 1 mg to | Approximately 1 mg |
| | Approximately 4 mg | |
| Vitamin D₃ | Approximately 2000 units | Approximately 5000 units |
| | (IU) to Approximately 10000 | (IU), which is approximately |
| | units (IU), which is | 0.125 mg |
| | approximately 0.05 mg to | |
| | approximately 0.25 mg | |
| Vitamin C | Approximately 100 mg to | Approximately 300 mg |
| | Approximately 1000 mg | |
| N-Acetyl Cysteine (NAC) | Approximately 300 mg to | Approximately 600 mg |
| | Approximately 1200 mg | |
| Green Coffee Bean Extract | Approximately 10 mg to | Approximately 20 mg |
| | Approximately 50 mg | |
| Turmeric Concentrate | Approximately 100 mg to | Approximately 300 mg |
| | Approximately 1000 mg | |
| Phosphatidylserine (optional) | Approximately 200 mg to | Approximately 300 mg |
| | Approximately 300 mg | |
| Niacin (optional) | Approximately 250 mg to | Approximately 250 mg |
| | Approximately 500 mg | |

The components and respective amounts of the components in percentage (%) by weight of the compound are listed in Table 2 below.

TABLE 2

| | Preferred Amount Range | Most Preferred Amount |
|---|---|---|
| Component | (percent (%) by weight) | (percent (%) by weight) |
| Vitamin B12 | Approximately 0.048% by | Approximately 0.096% by |
| | weight to Approximately | weight |
| | 0.192% by weight | |
| Biotin | Approximately 7.2% by | Approximately 14.47% by |
| | weight to Approximately | weight |
| | 14.47% by weight | |
| Folate | Approximately 0.048% by | Approximately 0.048% by |
| | weight to Approximately | weight |
| | 0.192% by weight | |
| Vitamin D₃ | Approximately 0.002% by | Approximately 0.006% by |
| | weight to Approximately | weight |
| | 0.012% by weight | |
| Vitamin C | Approximately 4.82% by | Approximately 14.47% by |
| | weight to Approximately | weight |
| | 48.23% by weight | |
| N-Acetyl Cysteine (NAC) | Approximately 14.47% by | Approximately 28.94% by |
| | weight to Approximately | weight |
| | 57.88% by weight | |
| Green Coffee Bean Extract | Approximately 0.48% by | Approximately 0.96% by |
| | weight to Approximately 2.4% | weight |
| | by weight | |
| Turmeric Concentrate | Approximately 4.82% by | Approximately 14.47% by |
| | weight to Approximately | weight |
| | 48.23% by weight | |
| Phosphatidylserine (optional) | Approximately 9.64% by | Approximately 14.47% by |
| | weight to Approximately | weight |
| | 14.47% by weight | |
| Niacin (optional) | Approximately 12.06% by | Approximately 12.06% by |
| | weight to Approximately | weight |
| | 24.12% by weight | |

In a preferred embodiment, the compound listed in Table 1 and/or Table 2 is administered once per day to a patient. In another embodiment, the compound listed in Table 1 and/or Table 2 is divided in half and administered twice per day to a patient, a first half administered to the patient at a first time of day and the second half administered to the patient at a second time of day. In other embodiments, the compound listed in Table 1 and/or Table 2 is apportioned into a set of dosages and administered at a set of times, each dosage of the set of dosages having a one-to-one correspondence with each time of the set of times.

In a preferred embodiment, the compound listed in Table 1 and/or Table 2 is taken with food. In this way, any possible gastro intestinal side effects, which depend on the reaction of each patient, is reduced or eliminated. In this embodiment, the compound listed in Table 1 and/or Table 2 is administered to a patient with a first food intake of the day and the second half administered to the patient with the second food intake of the day. For example, in one embodiment, a first pill is administered to the patient at breakfast and a second pill is administered to the patient at lunch. Other suitable food intake intervals may be employed.

In a preferred embodiment, the second half of the compound listed in Table 1 and/or Table 2 is administered to the patient on or before 2 pm. In this way, possible insomnia side effects, which depend on the reaction of each patient, are reduced or eliminated. In other embodiments, other times may be employed.

It will be appreciated by those skilled in the art that modifications can be made to the embodiments disclosed and remain within the inventive concept. Therefore, this invention is not limited to the specific embodiments disclosed, but is intended to cover changes within the scope and spirit of the claims.

1. A compound for treating symptoms of multiple sclerosis comprising:
from approximately 1 milligram to approximately 4 milligrams of vitamin B12;
from approximately 150 milligrams to approximately 300 milligrams of biotin;
from approximately 1 milligram to approximately 4 milligrams of folate;
from approximately 2000 international units to approximately 10,000 international units of vitamin D₃;
from approximately 100 milligrams to approximately 1000 milligrams of vitamin C;
from approximately 300 milligrams to approximately 1200 milligrams of n-acetylcysteine;
from approximately 10 milligrams to approximately 50 milligrams of green coffee bean extract; and,
from approximately 100 milligrams to approximately 1000 milligrams of turmeric concentrate.

2. The compound of claim 1, further comprising from approximately 200 milligrams to approximately 1000 milligrams of phosphatidylserine.

3. The compound of claim 1, further comprising from approximately 250 milligrams to approximately 500 milligrams of niacin.

4. The compound of claim 1, further comprising:
from approximately 200 milligrams to approximately 1000 milligrams of phosphatidylserine; and,
from approximately 250 milligrams to approximately 500 milligrams of niacin.

5. The compound of claim 1, wherein from approximately 1 milligrams to approximately 4 milligrams of vitamin B12 is approximately 2 milligrams of vitamin B12.

6. The compound of claim 1, wherein from approximately 150 milligrams to approximately 300 milligrams of biotin is approximately 300 milligrams of biotin.

7. The compound of claim 1, wherein from approximately 1 milligram to approximately 4 milligrams of folate is approximately 1 milligram of folate.

8. The compound of claim 1, wherein from approximately 2000 international units to approximately 10,000 international units of vitamin D₃ is approximately 5000 international units of vitamin D₃.

9. The compound of claim 1, wherein from approximately 100 milligrams to approximately 1000 milligrams of vitamin C is approximately 300 milligrams of vitamin C.

10. The compound of claim 1, wherein from approximately 300 milligrams to approximately 1200 milligrams of n-acetylcysteine is approximately 600 milligrams of n-acetylcysteine.

11. The compound of claim 1, wherein from approximately 10 milligrams to approximately 50 milligrams of green coffee bean extract is approximately 20 milligrams of green coffee bean extract.

12. The compound of claim 1, wherein from approximately 100 milligrams to approximately 1000 milligrams of turmeric concentrate is approximately 300 milligrams of turmeric concentrate.

13. The compound of claim 2, wherein from approximately 200 milligrams to approximately 1000 milligrams of phosphatidylserine is approximately 300 milligrams of phosphatidylserine.

14. The compound of claim 3, wherein from approximately 250 milligrams to approximately 500 milligrams of niacin is approximately 250 milligrams of niacin.

15. The compound of claim 4, wherein from approximately 200 milligrams to approximately 1000 milligrams of phosphatidylserine is approximately 300 milligrams of phosphatidylserine; and, wherein from approximately 250 milligrams to approximately 500 milligrams of niacin is approximately 250 milligrams of niacin.

16. A method for treating symptoms of multiple sclerosis in a patient comprising the steps of:
providing a compound comprising:
from approximately 1 milligrams to approximately 4 milligrams of vitamin B12;
from approximately 150 milligrams to approximately 300 milligrams of biotin;
from approximately 1 milligram to approximately 4 milligrams of folate;
from approximately 2000 international units to approximately 10,000 international units of vitamin D₃;
from approximately 100 milligrams to approximately 1000 milligrams of vitamin C;
from approximately 300 milligrams to approximately 1200 milligrams of n-acetylcysteine;
from approximately 10 milligrams to approximately 50 milligrams of green coffee bean extract; and,
from approximately 100 milligrams to approximately 1000 milligrams of turmeric concentrate; and,
apportioning the compound into a set of dosages of the compound; and,
administering the set of dosages of the compound based on a set of times.

17. The method of claim 16, wherein the step of apportioning the compound into a set of dosages of the compound further comprises the step of apportioning the compound into one dose of the compound.

18. The method of claim 16, wherein the step of apportioning the compound into a set of dosages of the compound further comprises the step of apportioning the compound into a first dose and a second dose.

19. The method claim 18, wherein the step of administering the set of dosages of the compound based on a set of times further comprises the steps of:
administering the first dose at a first time; and
administering the second dose at a second time.

20. The method of claim 16, wherein the step of administering the set of dosages of the compound based on a set of times further comprises the step of administering the set of dosages with a set of food intakes.

21. A compound for treating symptoms of multiple sclerosis comprising:
from approximately 0.048 percent by weight to approximately 0.192 percent by weight of vitamin B12;
from approximately 7.2 percent by weight to approximately 14.47 percent by weight of biotin;
from approximately 0.048 percent by weight to approximately 0.192 percent by weight of folate;
from approximately 0.002 percent by weight to approximately 0.012 percent by weight of vitamin D₃;
from approximately 4.82 percent by weight to approximately 48.23 percent by weight of vitamin C;
from approximately 14.47 percent by weight to approximately 57.88 percent by weight of n-acetylcysteine;
from approximately 0.48 percent by weight to approximately 2.4 percent by weight of green coffee bean extract; and,
from approximately 4.82 percent by weight to approximately 48.23 percent by weight of turmeric concentrate.

22. The compound of claim 21, further comprising from approximately 9.64 percent by weight to approximately 14.47 percent by weight of phosphatidylserine.

23. The compound of claim 21, further comprising from approximately 12.06 percent by weight to approximately 24.12 percent by weight of niacin.

24. The compound of claim 21, further comprising:
from approximately 9.64 percent by weight to approximately 14.47 percent by weight of phosphatidylserine; and,
from approximately 12.06 percent by weight to approximately 24.12 percent by weight of niacin.

25. The compound of claim 21, wherein from approximately 0.048 percent by weight to approximately 0.192 percent by weight of vitamin B12 is approximately 0.096 percent by weight of vitamin B12.

26. The compound of claim 21, wherein from approximately 7.2 percent by weight to approximately 14.47 percent by weight of biotin is approximately 14.47 percent by weight of biotin.

27. The compound of claim 21, wherein from approximately 0.048 percent by weight to approximately 0.192 percent by weight of folate is approximately 0.048 percent by weight of folate.

28. The compound of claim 21, wherein from approximately 0.002 percent by weight to approximately 0.012 percent by weight of vitamin D₃ is approximately 0.006 percent by weight of vitamin D₃.

29. The compound of claim 21, wherein from approximately 4.82 percent by weight to approximately 48.23 percent by weight of vitamin C is approximately 14.47 percent by weight of vitamin C.

30. The compound of claim 21, wherein from approximately 14.47 percent by weight to approximately 57.88 percent by weight of n-acetylcysteine is approximately 28.94 percent by weight of n-acetylcysteine.

31. The compound of claim 21, wherein from approximately 0.48 percent by weight to approximately 2.4 percent by weight of green coffee bean extract is approximately 0.96 percent by weight of green coffee bean extract.

32. The compound of claim 21, wherein from approximately 4.82 percent by weight to approximately 48.23 percent by weight of turmeric concentrate is approximately 14.47 percent by weight of turmeric concentrate.

33. The compound of claim 22, wherein from approximately 9.64 percent by weight to approximately 14.47 percent by weight of phosphatidylserine is approximately 14.47 percent by weight of phosphatidylserine.

34. The compound of claim 23, wherein from approximately 12.06 percent by weight to approximately 24.12 percent by weight of niacin is approximately 12.06 percent by weight of niacin.

35. The compound of claim 24, wherein from approximately 9.64 percent by weight to approximately 14.47 percent by weight of phosphatidylserine is approximately 14.47 percent by weight of phosphatidylserine; and, wherein from approximately 12.06 percent by weight to approximately 24.12 percent by weight of niacin is approximately 12.06 percent by weight of niacin.
